(19) United States
(12) Patent Application Publication
(10) Pub. No.: US 2004/0040050 A1
(43) Pub. Date: Feb. 26, 2004

(54) Production of agricultural animals from embryonic stem (es) cells

(76) Inventor: Michael D West, Boston, MA (US)

Correspondence Address: CROWELL & MORING LLP, INTELLECTUAL PROPERTY GROUP, P.O. BOX 14300, WASHINGTON, DC, 20044-4300, US

(21) Appl. No.: 10/276,001

(22) PCT Filed: May 10, 2001
(86) PCT No.: PCT/US01/15075

(30) Foreign Application Priority Data
May 10, 2000 (US) 09/567437

(51) Int. Cl. A01K 67/027; G06F 17/60

(52) U.S. Cl. 800/14; 800/19; 705/1; 119/300

(57) ABSTRACT

This invention describes a novel method of agricultural animal production that provides animals with optimum growth characteristics in a reliable and cost-effective manner. The invention utilizes a novel combination of Nuclear Transfer (NT) and Embryonic Stem (ES) cell technologies that improves the efficiency of delivering optimized animals and facilitates the introduction of genetic modifications into farm animals.

### FIELD OF THE INVENTION

This invention provides a novel method of producing animals, preferably agricultural animals, stem cell lines and endangered animals, by using a combination of nuclear transfer and embryonic stem cell techniques.

### BACKGROUND

The field of this invention is agriculture, more specifically animal agriculture and a means of improving the quality and reliability of the breeding and growing industry. At present farm animals, including, but not limited to poultry, pigs, and cattle, are produced by breeding from select breeding stock. This invention describes a means of capturing the genotype of superior animals by NT, and producing ES cells that can be easily cryopreserved and subsequently used to produce the final cloned animal for human consumption.

1. Nuclear Transfer

The first successful transfer of a nucleus from an adult mammary gland cell into an enucleated oocyte was reported in 1996 (Campbell et al., Nature 380: 64-6 (1996)). Nuclear transfer (NT) involves preparing a cytoplast as a recipient cell. In most cases, the cytoplast is derived from a mature metaphase II oocyte, from which the chromosomes have been removed. A donor cell nucleus is then placed between the zona and the cytoplast. Fusion and cytoplast activation are initiated by electrical stimulation. Successful reprogramming of the donor cell nucleus by the cytoplast is critical, and is a step which may be influenced by cell cycle (Wolf et al., Biol. Reprod. 60: 199-204 (1999)).

A number of pregnancies have been established using fetal cells as the source of donor nuclei. However, animal cloning is facilitated by the use of cell lines to create transgenic animals, which allow for the genetic manipulation of the cells in vitro before nuclear transfer. Id. The mechanisms regulating early embryonic development may be conserved among mammalian species, such that, for example, a bovine oocyte cytoplasm can support the introduced, differentiated, donor nucleus regardless of chromosome number, species or age of the donor fibroblast (Dominko et al., Biol. Reprod. 60: 1496-1502 (1999)).

Actively dividing fetal fibroblasts can be used as nuclear donors according to the procedure described in Cibelli et al., Science 280: 1256-9 (1998). Additional methods of preparing recipient oocytes for nuclear transfer of donor differentiated nuclei can be performed as described in International PCT Application Nos. 99/05266; 99/01164; 99/01163; 98/3916; 98/30683; 97/41209; 97/07668; 97/07669; and U.S. Pat. No. 5,843,754. Typically the transplanted nuclei are obtained from cultured embryonic stem (ES) cells, embryonic germ (EG) cells or other embryonic cells (See, e.g., International PCT Applications Nos. 95/17500 and 95/10599. Canadian Patent No. 2,092,258; Great Britain Patent No. 2.265.909; and U.S. Pat. Nos. 5,453,366; 5,057,420; 4,994,384; and 4,664,097). Inner cell mass (ICM) cells can also be used as nuclear donors (Sims et al., Proc. Natl. Acad. Sci. USA 90: 6143-7 (1990); and Keefer et al., Biol. Reprod. 50: 935-9 (1994).

II. Preparing Somatic Cells for Nuclear Transplantation or Nuclear Transfer

For purposes of animal husbandry, nuclear transfer can be used with embryonic stem cells (ES), inner cell mass cells (ICMs) and somatic cells.

Embryonic Stem Cells. Another system for producing transgenic animals has been developed that uses ES cells. In mice, ES cells have enabled researchers to select for transgenic cells and perform gene targeting. This method allows more genetic engineering than is possible with other transgenic techniques. For example, ES cells are relatively easy to grow as colonies in vitro, can be transfected by standard procedures, and the transgenic cells clonally selected by antibiotic resistance (Doetschman, “Gene transfer in embryonic stem cells.” IN TRANSGENIC ANIMAL TECHNOLOGY: A LABORATORY HANDBOOK 115-146 (C. Pinkert, ed., Academic Press. Inc., New York 1994)). Furthermore, the efficiency of this process is such that sufficient transgenic colonies (hundreds to thousands) can be produced to allow a second selection for homologous recombinants (Id.). ES cells can then be combined with a normal host embryo and, because they retain their potency, can develop into all the tissues in the resulting chimeric animal, including the germ cells. Thus, the transgenic modification is transmissible to subsequent generations.

Methods for deriving embryonic stem (ES) cell lines in vitro from early preimplantation mouse embryos are well known (Evans et al., Nature 29: 154-6 (1981); and Martin, Proc. Natl. Acad. Sci. USA 78: 7634-8 (1981)). ES cells can be passaged in an undifferentiated stale, provided that a feeder layer of fibroblast cells (Evans et al. 1981) or a differentiation inhibiting source (Smith et al., Dev. Biol. 121: 1-9 (1987)), is present.

In view of their ability to transfer their genome to the next generation, ES cells have potential utility for germ line manipulation of livestock animals. Some research groups have reported the isolation of pluripotent embryonic cell lines. For example, Notarianni et al., J. Reprod. Fert. Suppl. 43: 55-260 (1991) reported the establishment of stable, pluripotent cell lines from pig and sheep blastocysts, which exhibit some morphological and growth characteristics similar to that of cells in primary cultures of inner cell masses (ICMs) isolated immunosurgically from sheep blastocysts. Also, Notarianni et al., J. Reprod. Fert. Suppl. 41: 51-56 (1990) disclosed maintenance and differentiation in culture of putative pluripotent embryonic cell lines from pig blastocysts. Gerfen et al., Anim. Biotech. 6: 1-14 (1995) disclosed the isolation of embryonic cell lines from porcine blastocysts, which do not require mouse embryonic fibroblast feeder layers and reportedly differentiate into several different cell types during culture.

Further, Saito et al., Roux's Arch. Dev. Biol. 201: 134-41 (1992) reported cultured, bovine embryonic stem cell-like cell lines, which survived three passages, but were lost after the fourth passage. Handyside et al., Roux's Ach. Dev. Biol. 196: 185-90 (1987) disclosed culturing immunosurgically isolated sheep embryo ICMs under conditions that allow for the isolation of mouse ES cell lines derived from mouse ICMs.

Campbell et al., Nature 380: 64-6 (1996) reported the production of live lambs following nuclear transfer of cultured embryonic disc (ED) cells from day nine ovine embryos cultured under conditions which promote the isolation of ES cell lines in the mouse.

Purportedly, animal stem cells have been isolated, selected and propagated for use in obtaining transgenic animals (see Evans et al., WO 90/03432; Smith et al., WO 94/24274: and Wheeler et al., WO 94/26884). Evans et al., also reported the derivation of purportedly pluripotent ES cells from porcine and bovine species, which purportedly are useful for the production of transgenic animals.

ES cells from a transgenic embryo can be used in nuclear transplantation. The use of ungulate ICM cells for nuclear trans-plantation also has been reported. In the case of live-stock animals (e.g. ungulates) nuclei from similar preimplantation livestock embryos support the development of enucleated oocytes to term (Keefer el a). Biol. Reprod. 50: 935-39 (1994); Smith et al., Biol. Reprod. 40: 1027-1035 (1989)). In contrast, nuclei from mouse embryos do not support development of enucleated oocytes beyond the eight-cell stage after transfer (Cheong et al., Biol. Reprod. 48: 958-63 (1993)). Therefore. ES cells from livestock animals are highly desirable, because they may provide a potential source of totipotent donor nuclei, genetically manipulated or other-wise, for nuclear transfer procedures.

Use of ICM Cells. Collas et al., Mol. Reprod. Dev. 38: 264-7 (1994) disclosed nuclear transplantation of bovine ICMs by microinjection of the lysed donor cells into enucleated mature oocytes. Culturing of embryos in vitro for seven days produced fifteen blastocysts which, upon transfer into bovine recipients, resulted in four pregnancies and two births. Also, Keefer et al. (1994) disclosed the use of bovine ICM cells as donor nuclei in nuclear transfer procedures, to produce blastocysts which also resulted in several live offspring. Further, Sims et al., Proc. Natl. Acad. Sci. USA 90: 6143-7 (1993) disclosed the production of calves by transfer of nuclei from short-term in vitro cultured bovine ICM cells into enucleated mature oocytes.

Therefore, notwithstanding what has previously been reported in the literature, there exists a need for improved methods of obtaining transgenic animals by faster more cost effective means. The invention also provides for methods of making such animals and embryonic stem cell lines.

### SUMMARY OF THE INVENTION

It is an object of the invention to provide an animal produced from embryonic stem cells. Where such embryonic stem cells original from a cloned embryo. The animal can be a mammal or an avian, and preferably a farm animal. Other animals contemplated for production by the methods disclosed include: bovines, non-human primates, ovines, murines, porcines, canines, felines, or caprines.

It is a further objection of the invention to provide ES cells produced from an embryo made by nuclear transfer.

Another objection of the invention provides for a business model whereby cryopreserved clonal ES cells are marketed instead of live animals for the production of farm animals.

Still a further object of the invention provides for a method for producing an embryonic stem (ES) derived cloned mammal comprising the following steps: (i) isolating a somatic cell from an animal having desired characteristic(s); (ii) transfecting such cell with a marker that allows for cells containing to be selected by positive selection; (iii) using said transfected cell as a cell or nuclear donor during a nuclear procedure; (iv) culturing the resultant nuclear transfer embryo under conditions that result into development into a blastocyst or post-blastocyst stage embryo. (v) isolating totipotent (e.g. inner cell mass cells) from said embryo and expanding said cells in culture to produce ES cells; (vi) optionally cryopreserving said expanded ES cells; (vii) inserting said ES cells into a host embryo of 1 to 200 cells which is not resistant to the selectable marker; (viii) culturing the resultant embryo under selective conditions for the selectable marker to obtain embryos that substantially consist of cells that comprise genome of ES cells; and (ix) after embryos have reached desired size transferring said embryo to a recipient female.

Also disclosed is A method for deriving a cloned animal from an ES cell comprising: (i) isolating a somatic cell from an animal having desired characteristics; (ii) using said cell as a cell or nuclear donor during nuclear transfer; (iii) using the resultant nuclear transfer fusion to produce an embryo of the blastocyst stage or later; (iv) isolating totipotent cells (e.g., inner cell mass cells) from said embryo and expanding said cells in culture to produce ES cells; (v) optionally cryopreserving said ES cells; (vi) inserting some of said ES cells into a host embryo of 2 to 200 cells which is incapable of development; (vii) culturing the resultant embryo until it is of a size suitable for implantation into a recipient female; (viii) transferring said cultured embryo into a recipient female.

Another object of the invention is to provide for a method for producing an avian from ES cells comprising: (i) isolating ES cells from an avian having desired characteristics; (ii) expanding said ES cells in culture and optionally cryopreserving said expanded ES cells; (iii) obtaining eggs that are unable to develop into an embryo; (iv) injecting said eggs with said ES cells: and (g) incubating said eggs to produce avian offspring having the genotype of ES cells. Preferred avian species include: chicken, turkey, guinea hen, ostrich, eagle, osprey, condor, bird of prey, or avian near extinction.

Another object of the invention is to provide for animals and ES cells in which one or more genes have been genetically introduced, deleted or otherwise modified.

### BRIEF DESCRIPTION OF THE FIGURES

FIG. 1. Generation of transgenic ES-like cells. FIG. 1A: Embryo-derived ES-like cells. FIG. 1B. β-galactosidase activity of transgenic embryo-derived ES-like cells. FIG. 1C. β-galactosidase activity of transgenic fetal fibroblasts. FIG. 1D. Nuclear transfer-derived ES-like cells. PCR ethidium bromide gel of β-galactosidase fragment. Lane 1: non-transgenic embryo-derived ES-like cells lane 2: transgenic embryo-derived ES-like cells; lane 3: transgenic fetal fibroblasts lane 4: transgenic nuclear transfer-derived ES-like cells; lane 5: non-transgenic fetal fibroblasts; lane 8: template.

FIG. 2. Generation of transgenic ES-like cells (A) by microinjection and (B) by somatic cell nuclear transfer.

FIG. 3. Southern blot analysis of PCR-amplified products of tissues from chimeric calves. Calves 901 and 903 were generated from embryo-derived ES-like transgenic cells. Calves 907 and 912 were generated from nuclear transfer-derived ES-like transgenic cells.

FIG. 4. FISH analysis of spleen from calf 911 (FIG. 1A) produced with NT-derived ES-like cells, negative control spleen (FIG. 1B), testis of calf 903 produced with embryo-derived ES-like cells (FIG. 1C), and negative control testis (FIG. 1D).

### BRIEF DESCRIPTION OF THE INVENTION

The current technologies used to deliver optimized farm animals are based on selective breeding, and expansion from preferred breeding stocks. Animals that have been selected on the basis of superior characteristics, such as meal content, egg production (in the case of poultry), feed conversion ratio and so on are used to breed large numbers of animals that are in turn used in the human food supply. This traditional process has profound inherent inefficiencies. The phenotype observed in an individual animal is often only partially transmitted in the progeny of that animal. Therefore, traditional breeding schemes are inefficient in capturing the very best phenotype in all of the progeny animals. In addition, in some cases, such as in poultry breeding, the breeding stock need to be prepared months in advance of actual sale, and the demand at the end of the breeding process may be far above or below the numbers of animals actually produced. Therefore the breeder may experience large losses from overproduction or lost sales from underproduction that cannot be adequately controlled. This invention allows the production of the final animal from cloned embryonic stem cells that have the ideal phenotype and can be easily cryopreserved and thawed to meet the needs of the grower. In addition, embryonic stem cells possess replicative immortality facilitating the modification of the genome by gene targeting and other means, allowing the supplier of such cells to introduce genetic modifications into the germ line of the cloned animals to meet the needs of the marketplace.

### DETAILED DESCRIPTION

1. Definitions

By “animal” is meant to include avians, mammals, reptiles and amphibians. Preferred animals include avians and mammals as well as any animal that is an endangered species. Preferred birds include domesticated birds (e.g. quail, chickens. ducks, geese, turkeys, and guinea hens) as well as other birds such as birds of prey (e.g., hawks, falcons, ospreys, condors. etc.), endangered birds (e.g., parrots, California condor, etc.), ostriches etc. Preferred mammals include murine, caprine, ovine, bovine, porcine, canine, feline and primate. Of these, preferred members include domesticated ungulates (e.g., cattle, buffalo, pigs, sheep, and goats) and humans.

By “female surrogate” is meant a female animal into which an embryo of the invention is inserted for gestation. Typically, the female animal is of the same animal species as the embryo, but the female surrogate may also be of a different animal species. The embryo, as used herein, can include a complex of two or more cells.

By “cytoplast” is meant the fragment of the cell remaining once the nucleus is removed.

By “enucleated oocyte” is meant an animal egg which has had its endogenous nucleus removed or inactivated.

By “sperm” “semen,” “sperm sample,” and “semen sample” are meant the ejaculate from a male animal which contains spermatozoa. A mature sperm cell is a “spermatozoon,” whereas the precursor is a “spermatid.” Spermatids are the haploid products of the second meiotic division in spermatogenesis, which differentiate into spermatozoa.

The terms “nuclear transfer” or “nuclear transplantation” refer to a method of cloning, wherein the donor cell nucleus is transplanted into a cell before or after removal of its endogenous nucleus. The cytoplast could be from an enucleated oocyte, an enucleated ES cell, an enucleated EG cell, an enucleated embryonic cell or an enucleated somatic cell. Nuclear transfer techniques or nuclear transplantation techniques are known in the literature (Campbell et al., Theriogenology 43: 181 (1995); Collas et al., Mol. Reprod. Dev. 38: 264-267 (1994); Keefer et al., Biol. Reprod. 50: 935-939 (1994); Sims et al., Proc. Natl. Acad. Sci. USA 90: 6143-6147 (1993); Evans et al., WO 90/03432; Smith et al., WO 94/24274; and Wheeler et al., WO 94/26884. Also U.S. Pat. Nos. 4,994,384 and 5,057,420 describe procedures for bovine nuclear transplantation. In the subject application, “nuclear transfer” or “nuclear transplantation” or “NT” are used interchangeably.

The terms “nuclear transfer unit” and “NT unit” refer to the product of fusion between or injection of a somatic cell or cell nucleus and an enucleated cytoplast (e.g., an enucleated oocyte), which is some-times referred to herein as a fused NT unit.

By “somatic cell” is meant any cell of a multicellular organism, preferably an animal, that does not become a gamete.

By “differentiate” or “differentiation” is meant to refer to the process in development of an organism by which cells become specialized for particular functions. Differentiation requires that there is selective expression of portions of the genome.

By, “inner cell mass” or “ICM” is meant a group of cells found in the mammalian blastocyst that give rise to the embryo and are potentially capable of forming all tissues, embryonic and extra-embryonic, except the trophoblast.

By “feeder layer” is meant a layer of cells to condition the medium in order to culture other cells, particularly to culture those cells at low or clonal density.

By “medium” or “media” is meant the nutrient solution in which cells and tissues are grown.

II. Production of ES-Derived Cloned Mammals:

The Steps used are:

1) Isolate a somatic cell from an optimal animal

2) Tranfect such cells with an antibiotic resistance gene or any other gene that would allow the selection of these cells by positive selection. An example would be the use of the neomycin resistance gene.

3) Take one of these cells and perform nuclear transfer to be able to have ES cells from the somatic cells.

4) Cryopreserve large quantities of ampules of the ES cells.

5) Take some of these ES cells between 2 to 20, preferably 12 and inject them into a host mammalian embryo. Such embryo should be in the stage between one to 200 cells preferably between 8 to 16 cells. The host embryo should not be resistant to the previously-mentioned selectable marker gene.

6) Place the embryos is culture with a specific dosage of the selection substance, in the example of the neomycin resistance gene. G418.

7) After 7 days in culture, embryos are transferred into the recipient females.

III. An Alternative Method for the Production of ES-Derived Cloned Mammals

The Steps used are:

1) Isolate a somatic cell from an optimal animal.

2) Take one of these cells and perform nuclear transfer to be able to have ES cells from the somatic cells.

3) Cryopreserve large quantities of ampules of the ES cells.

4) Take some of these ES cells between 2 to 20, preferably 12 and inject them into a host mammalian embryo. Such embryo should be in the stare between one to 200 cells preferably between 8 to 16 cells. The host embryo should be incapable of development, being for example, a tetraploid embryo.

5) After 7 days in culture, embryos are transferred into the recipient females.

IV. Production of ES-Derived Poultry

The Steps used are:

1) Isolate ES cells from a superior breeding stock of avian species.

2) Scale cells up and cryopreserve large quantities of ampules of cells.

3) Recipient eggs are prepared that are deficient in embryo production by genetic modification of the laying hen, or by exogenous means such as irradiation.

4) injection of ES cells from the genotype desired.

5) Incubation to hatch.

6) Growth of final broiler.

V. Genetic Modification of Animal ES Cells

The marketed avian or mammalian ES cells may subsequently be modified by gene targeting or other means of genetic modification to introduce improved genetics of the final animal.

VI. Business Marketing Model

The product envisioned is cryopreserved animal ES cells that are stable for long periods of time, and can be stored inexpensively. When the final animal is in demand, the vials can he thawed and injected into the host embryo to produce the final animal. This allows the marketing of cryovials as opposed to live animals. The business model is to market clonal ES cells to the final growers. The end customer will also be sold the injection equipment and allied supplies such as host embryos and eggs to produce the final animals.

VII. Nuclear Transfer

Preferably, the NT units used to produce ES-like cells will be cultured to a size of at least 2 to 400 cells, preferably 4 to 128 cells, and most preferably to a size of at least about 50 cells.

In the present invention, embryonic stem cells, embryonic germ cells and embryonic stem-like cells can be produced according to the present invention. The present application refers to stem-like cells rather than stem cells because of the manner in which they are typically produced. i.e. by cross-species nuclear transfer. While these cells are expected to possess similar differentiation capacity as normal stem cells they may possess some insignificant differences because of the manner they are produced. For example, these stem-like cells may possess the mitochondria of the oocytes used for nuclear transfer, and thus not behave identically to conventional embryonic stem cells.

Based on the fact that human cell nuclei can be effectively transplanted into bovine oocytes, it is reasonable to expect that human cells maybe transplanted into oocytes of other non-related species, e.g. other ungulates as well as other animals. In particular, other ungulate oocytes should be suitable, e.g., pigs, sheep, horses, goats. etc. Also, oocytes from other sources should be suitable e.g. oocytes derived from other primates, amphibians, rodents, rabbits, guinea pigs, etc. Further, using similar methods, it should be possible to transfer human cells or cell nuclei into human oocytes and use the resultant blastocysts to produce human ES cells.

Therefore, in one embodiment, the present invention involves the transplantation of an animal or human cell nucleus or animal or human cell into an oocyte (preferably enucleated) of an animal species different from the donor nuclei, by injection or fusion. To produce an NT unit containing cells which may be used to obtain embryonic or stem-like cells and/or cell cultures. In another embodiment, a nucleus of an animal is injected or fused to an oocyte from the same animal species.

Enucleation (removal of endogenous oocyte nucleus) may be effected before or after nuclear transfer. For example, the invention may involve the transplantation of an ungulate cell nucleus or ungulate cell into an enucleated oocyte, e.g. another ungulate or non-ungulate, by injection or fusion, which cells and/or nuclei are combined to produce NT units and which are cultured under conditions suitable to obtain multicellular NT units, preferably comprising at least about 2 to 400 cells, more preferably 4 to 128 cells, and most preferably at least about 50 cells. The cells of such NT units may be used to produce EG cells, ES cells, and ES-like cells as well as cell colonies upon culturing.

Nuclear transfer techniques or nuclear transplantation techniques are known in the literature and are described in many of the references cited in the Background of the Invention. See, in particular, Campbell et al. Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci. USA, 90:6143-6147 (1993); WO 94/26884; WO 94/24274, and WO 90/03432, which are incorporated by reference in their entirety herein. Also, U.S. Pat. Nos. 4,944,384 and 5,057,420 describe procedures for bovine nuclear transplantation. See, also Cibelli et al, Science, Vol. 280:1256-1258 (1998).

Human or animal cells, preferably mammalian cells, may be obtained and cultured by well known methods. Human and animal cells useful in the present invention include, by way of example, epithelial, neural cells, epidermal cells, keratinocytes, hematopoietic cells, melanocytes, chondrocytes, lymphocytes (B and T lymphocytes), other immune cells, erythocytes, macrophages, melanocytes, monocytes, mononuclear cells, fibroblasts, cardiac muscle cells, and other muscle cells. etc. Moreover, the human cells used for nuclear transfer may be obtained from different organs. e.g. skin, lung, pancreas, liver, stomach, intestine, heart, reproductive organs, bladder, kidney, urethra and other urinary organs. etc. These are just examples of suitable donor cells. Suitable donor cells, i.e., cells useful in the subject invention, may be obtained from any cell or organ of the body. This includes all somatic or germ cells. Preferably, the donor cells or nucleus would comprise actively dividing. i.e. non-quiescent., cells as this has been reported to enhance cloning efficacy. Also preferably, such donor cells will be in the G1 cell cycle.

The resultant blastocysts may be used to obtain embryonic stem cell lines according to the culturing methods reported by Thomson et al., Science, 282:1145-1147 (1998) and Thomson et al., Proc. Natl. Acad. Sci., USA, 92:7544-7848 (1995), incorporated by reference in their entirety herein.

In the Example which follows, the cells used as donors for nuclear transfer were epithelial cells derived from the oral cavity of a human donor and adult human keratinocytes. However, as discussed, the disclosed method is applicable to other human cells or nuclei. Moreover, the cell nuclei may be obtained from both human somatic and germ cells.

It is also possible to arrest donor cells at mitosis before nuclear transfer, using a suitable technique known in the art. Methods for stopping the cell cycle at various stages have been thoroughly reviewed in U.S. Pat. No. 5,262,409, which is herein incorporated by reference. In particular, while cycloheximide has been reported to have an inhibitory effect on mitosis (Bowen and Wilson (1955) J. Heredity, 45:3-9), it may also be employed for improved activation of mature bovine follicular oocytes when combined with electric pulse treatment (Yang et al., (1992) Biol. Reprod., 42 (Suppl. 1): 117).

Zygote gene activation is associated with hyperacetylation of Histone H4. Trichostatin-A has been shown to inhibit histone deacetylase in a reversible manner (Adenot et al., Development (1997) 124(22): 4615-4625; Yoshida et al., Bioessays (1995) 17(5): 423-430), as have other compounds. For instance, butyrate is also believed to cause hyper-acetylations of histones by inhibiting histone deacetylase. Generally, butyrate appears to modify gene expression and in almost all cases its addition to cells in culture appears to arrest cell growth. Use of butyrate in this regard is described in U.S. Pat. No. 5,681,718, which is herein incorporated by reference. Thus, donor cells may be exposed to Trichostatin-A or another appropriate deacetylase inhibitor prior to fusion, or such a compound may be added to the culture media prior to genome activation.

Additionally, demethylation of DNA is thought to be a requirement for proper access of transcription factors to DNA regulatory sequences. Global demethylation of DNA from the eight-cell stage to the blastocyst stage in preimplantation embryos has previously been described (Stein et al., Mol. Reprod. & Dev., 47(4): 421-429). Also, Jaenisch et al. (1997) have reported that 5-azacytidine can be used to reduce the level of DNA methylation in cells, potentially leading to increased access of transcription factors to DNA regulatory sequences. Accordingly, donor cells may be exposed to 5-azacytidine (5-Aza) previous to fusion, or 5-Aza may be added to the culture medium from the 8 cell stage to blastocyst. Alternatively, other known methods for effecting DNA demethylation may be used.

The oocytes used for nuclear transfer may be obtained from animals including mammals, avians, reptiles, and amphibians. Suitable mammalian sources for oocytes include sheep, bovines, ovines, pigs, horses, rabbits, goats, guinea pigs, mice, hamsters, rats, primates, humans, etc. In the preferred embodiments, the oocytes will be obtained from primates or ungulates, e.g., a bovine.

Methods for isolation of oocytes are well known in the art. Essentially, this will comprise isolating oocytes from the ovaries or reproductive tract of a mammal or amphibian, e.g., a bovine. A readily available source of bovine oocytes is slaughterhouse materials.

For the successful use of techniques such as genetic engineering, nuclear transfer and cloning, oocytes must generally be matured in vitro before these cells may be used as recipient cells for nuclear transfer, and before they can be fertilized by the sperm cell to develop into an embryo. This process generally requires collecting immature (prophase I) oocytes from animal ovaries. e.g. bovine ovaries obtained at a slaughterhouse and maturing the oocytes in a maturation medium prior to fertilization or enucleation until the oocytes attains the metaphase II stage, which in the case of bovine oocytes generally occurs about 18-24 hours post-aspiration. For purposes of the present invention, this period of time is known as the “maturation period.” As used herein for calculation of time periods. “aspiration” refers to aspiration of the immature oocyte from ovarian follicles.

Additionally, metaphase II stage oocytes, which have been matured in vivo have been successfully used in nuclear transfer techniques. Essentially, mature meta-phase II oocytes are collected surgically from either non-superovulated or superovulated cows or heifers 35 to 48 hours past the onset of estrus or past the injection of human chorionic gonadotropin (hCG) or similar hormone.

The stage of maturation of the oocyte at enucleation and nuclear transfer has been reported to be significant to the success of NT methods. (See, e.g., Prather et al., Differentiation: 48: 1-8, 1991). In general, previous successful mammalian embryo cloning practices used metaphase II stage oocyte as the recipient oocyte because at this stage it is believed that the oocyte can be or is sufficiently “activated” to treat the introduced nucleus as it does a fertilizing sperm. In domestic animals, and especially cattle, the oocyte activation period generally ranges from about 16-52 hours, preferably about 28-42 hours post-aspiration.

For example, immature oocytes may be washed in HEPES buffered hamster embryo culture medium (HECM) as described in Seshagine et al., Biol. Reprod., 40: 544-606 (1989) and then placed into drops of maturation medium consisting of 50 μl of tissue culture medium (TCM) 199 containing 10% fetal calf serum which contains appropriate gonadotropins such as luteinizing hormone (LH) and follicle stimulating hormone (FSH), and estradiol under a layer of lightweight paraffin or silicon at 39° C.

After a fixed time maturation period, which typically will range from about 10 to 40 hours, and preferably about 16-18 hours, the oocytes will typically be enucleated. Prior to enucleation the oocytes will preferably be removed and placed in HECM containing 1 mg/ml of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows. As noted above, enucleation may be effected before or after introduction of donor cell or nucleus because the donor nucleus is readily discernible from endogenous nucleus.

Enucleation may be effected by known methods, such as described in U.S. Pat. No. 4,994,384 which is incorporated by reference herein. For example, metaphase II oocytes are either placed in HECM, optionally containing 7.5 μg/ml cytochalasin B, for immediate enucleation, or may be placed in a suitable medium, for example CR1aa, plus 10% estrus cow serum, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify, those of which have been successfully enucleated. This screening may be effected by staining the oocytes with 1 μg/ml 33342 Hoechst dye in HECM, and then viewing the oocytes under ultraviolet irradiation for less than 10 seconds. The oocytes that have been successfully enucleated can then be placed in a suitable culture medium.

In the present invention, the recipient oocytes will typically be enucleated at a time ranging from about 10 hours to about 40 hours after the initiation of in vitro maturation, more preferably from about 16 hours to about 24 hours after initiation of in vitro maturation, and most preferably about 16-18 hours after initiation of in vitro maturation. Enucleation may be effected before, simultaneous or after nuclear transfer. Also, enucleation may be effected before, after or simultaneous to activation.

A single animal or human cell or nucleus derived therefrom which is typically heterologous to the enucleated oocyte will then be transferred into the perivitelline space of the oocyte, typically enucleated, used to produce the NT unit. However, removal of endogenous nucleus may alternatively be effected after nuclear transfer. The animal or human cell or nucleus and the enucleated oocyte will be used to produce NT units according to methods known in the art. For example, the cells may be fused by electrofusion. Electrofusion is accomplished by providing a pulse of electricity that is sufficient to cause a transient break down of the plasma membrane. This breakdown of the plasma membrane is ver, short because the membrane reforms rapidly. Essentially, if two adjacent membranes are induced to break down, upon reformation the lipid bilayers intermingle and small channels will open between the two cells. Due to the thermodynamic instability of such a small opening, it enlarges until the two cells become one. Reference is made to U.S. Pat. No. 4,997,384, by Prather et a)., (incorporated by reference in its entirety herein) for a further discussion of this process. A variety of electrofusion media can be used including, e.g., sucrose, mannitol, sorbitol and phosphate buffered solution. Fusion can also be accomplished using Sendai virus as a fusogenic agent (Graham, Wister Inot. Symp. Monogr., 9:19, 1969).

Also, in some cases (e.g. with small donor nuclei) it may be preferable to inject the nucleus directly into the oocyte rather than using electroporation fusion. Such techniques are disclosed in Collas and Barnes, Mol. Reprod. Dev., 38: 264-7 (1994), and incorporated by reference in its entirety herein.

Preferably, the human or animal cell and oocyte are electrofused in a 500 μm chamber by application of an electrical pulse of 90-120V for about 15 μsec, about 24 hours after initiation of oocyte maturation. After fusion, the resultant fused NT units are preferably placed in a suitable medium until activation. e.g. one identified infra. Typically activation will be effected shortly thereafter, typically less than 24 hours later, and preferably about 4-9 hours later. However, it is also possible to activate the recipient oocyte before or proximate (simultaneous) to nuclear transfer, which may or may not be enucleated. For example, activation may be effected from about twelve hours prior to nuclear transfer to about twenty-four hours after nuclear transfer. More typically, activation is effected simultaneous or shortly after nuclear transfer, e.g., about four to nine hours later.

The NT unit may be activated by known methods. Such methods include, e.g., culturing the NT unit at sub-physiological temperature, in essence by applying a cold, or actually cool temperature shock to the NT unit. This may be most conveniently done by culturing the NT unit at room temperature, which is cold relative to the physiological temperature conditions to which embryos are normally exposed.

Alternatively, activation may be achieved by application of known activation agents. For example, penetration of oocytes by sperm during fertilization has been shown to activate prefusion oocytes to yield greater numbers of viable pregnancies and multiple genetically identical calves after nuclear transfer. Also, treatments such as electrical and chemical shock or cycloheximide treatment may also be used to activate NT embryos after fusion. Suitable oocyte activation methods are the subject of U.S. Pat. No. 5,496,720, to Susko-Parrish et al. which is herein incorporated by reference.

For example, oocyte activation may be effected by simultaneously or sequentially:

(i) increasing levels of divalent cations in the oocyte, and

(ii) reducing phosphorylation of cellular proteins in the oocyte.

This will generally be effected by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators.

Phosphorylation may be reduced by known methods, e.g. by the addition of kinase inhibitors, e.g., serine-threonine kinase inhibitors, such as 6-dimethylamino-purine, staurosporine, 2-aminopurine, and sphingosine.

Alternatively, phosphorylation of cellular proteins may be inhibited by introduction of a phosphatase into the oocyte, e.g., phosphatase 2A and phosphatase 2B.

Specific examples of activation methods are listed below.

1. Activation by Ionomycin and DMAP

1—Place oocytes in Ionomycin (5 μM) with 2 mM of DMAP for 4 minutes;

2—Move the oocytes into culture media with 2 mM of DMAP for 4 hours;

3—Rinse four times and place in culture.

2. Activation by Ionomycin DMAP and Roscovitin

1—Place oocytes in Ionomycin (5 μM) with 2 mM of DMAP for four minutes;

2—Move the oocytes into culture media with 2 mM of DMAP and 200 microM of Roscovitin for three hours.

3—Rinse four times and place in culture.

3. Activation by exposure to Ionomycin followed by cytochalasin and cycloheximide.

1—Place oocytes in Ionomycin (5 microM) for four minutes;

2—Move oocytes to culture media containing 5 μg/ml of cytochalasin B and 5 μg/ml of cycloheximide for five hours;

3—Rinse four times and place in culture.

4. Activation by electrical pulses

1—Place eggs in mannitol media containing 100 μM CaCL₂;

2—Deliver three pulses of 1.0 kVcm⁻¹ for 20 μsec. each pulse 22 minutes apart;

3—Move oocytes to culture media containing 5 μg/ml of cytochalasin B for three hours.

5. Activation by exposure with ethanol followed by cytochalasin and cycloheximide

1—Place oocytes in 7% ethanol for one minute;

2—Move oocytes to culture media containing 5 μg/ml of cytochalasin B and 5 μg/ml of cycloheximide for five hours;

3—Rinse four times and place in culture.

6. Activation by microinjection of adenophostine

1—Inject oocytes with 10 to 12 picoliters of a solution containing 10 μM of adenophostine;

2—Put oocytes in culture.

7. Activation by microinjection of sperm factor

1—Inject oocytes with 10 to 12 picoliters of sperm factor isolated, e.g., from primates, pigs, bovine, sheep, goals, horses, mice, rats, rabbits or hamsters.

2—Put eggs in culture.

8. Activation by microinjection of recombinant sperm factor.

9. Activation by Exposure to DMAP followed by Cycloheximide and Cytochalasin B

Place oocytes or NT units, typically about 22 to 28 hours post maturation in about 2 mM DMAP for about one hour, followed by incubation for about two to twelve hours, preferably about eight hours, in 5 μg/ml of cytochalasin B and 20 μg/ml cycloheximide.

The above activation protocols are exemplary of protocols used for nuclear transfer procedures, e.g., those including the use of primate or human donor cells or oocytes. However, the above activation protocols may be used when either or both the donor cell and nucleus is of ungulate origin, e.g., a sheep, buffalo, horse, goat, bovine, pig and/or wherein the oocyte is of ungulate origin, e.g. sheet, pig, buffalo, horse, goal, bovine, etc., as well as for other species.

As noted, activation may be effected before, simultaneous, or after nuclear transfer. An general, activation will be effected about 40 hours prior to nuclear transfer and fusion to about 40 hours after nuclear transfer and fusion, more preferably about 24 hours before 10 about 24 hours after nuclear transfer and fusion, and most preferably from about 4 to 9 hours before nuclear transfer and fusion to about 4 to 9 hours after nuclear transfer and fusion. Activation is preferably effected after or proximate to in vitro or in vivo maturation of the oocyte, e.g., approximately simultaneous or within about 40 hours of maturation, more preferably within about 24 hours of maturation.

Activated NT units may be cultured in a suitable in vitro culture medium until the generation of embryonic or stem-like cells and cell colonies. Culture media suitable for culturing and maturation of embryos are well known in the art. Examples of known media, which may be used for bovine embryo culture and maintenance. include Ham's F-10 supplemented with 10% fetal calf serum (FCS), Tissue Culture Medium-199 (TCM-199) supplemented with 10% fetal calf serum. Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% fetal calf serum, 0.2 M pyruvate and 50 μg/ml gentamycin sulphate. Any of the above may also involve co-culture with a variety of cell types such as granulosa cells oviduct cells. BRL cells and uterine cells and STO cells.

In particular, human epithelial cells of the endometrium secrete leukemia inhibitory factor (LIF) during the preimplantation and implantation period. Therefore, the addition of LIF to the culture medium could be of importance in enhancing the in vitro development of the reconstructed embryos. The use of LIF for embryonic or stem-like cell cultures has been described in U.S. Pat. No. 5,712,156, which is herein incorporated by reference.

Another maintenance medium is described in U.S. Pat. No. 5,096,822 to Rosenkrans, Jr. et al., which is incorporated herein by reference. This embryo medium, named CR1, contains the nutritional substances necessary to support an embryo. CR1 contains hemicalcium L-lactate in amounts ranging from 1.0 mM to 10 mM, preferably 1.0 mM to 5.0 mM. Hemicalcium L-lactate is L-lactate with a hemicalcium salt incorporated thereon.

Also, suitable culture medium for maintaining human embryonic cells in culture as discussed in Thomson et al., Science, 282:1145-7 (1998) and Proc. Natl. Acad. Sci. USA, 92: 7844-8 (1995).

Afterward, the cultured NT unit or units are preferably washed and then placed in a suitable media, e.g., CR1aa medium. Ham's F-10, Tissue Culture Media-199 (TCM-199). Tyrodes-Albumin-Lactate-Pyruvate (TALP) Dulbecco's Phosphate Buffered Saline (PBS), Eagle's or Whitten's, preferably containing about 10% FCS. Such culturing will preferably be effected in well plates which contain a suitable confluent feeder layer. Suitable feeder layers include, by way of example, fibroblasts and epithelial cells e.g. fibroblasts and uterine epithelial cells derived from ungulates, chicken fibroblasts murine (e.g. mouse or rat) fibroblasts. STO and S1-m220 feeder cell lines, and BRL cells.

In the preferred embodiment, the feeder cells will comprise mouse embryonic fibroblasts. Means for preparation of a suitable fibroblast feeder layer are described in the example which follows and is well within the skill of the ordinary artisan.

The NT units are cultured on the feeder layer until the NT units reach a size suitable for obtaining cells which may be used to produce embryonic stem-lire cells or cell colonies. Preferably, these N units will be cultured until they reach a size of at least about 2 to 400 cells, more preferably about 4 to 128 cells, and most preferably at least about 50 cells. The culturing will be effected under suitable conditions. i.e. about 38.5° C. and 5% CO₂ with the culture medium changed in order to optimize growth typically about every 2-5 days, preferably about every 3 days.

In the case of human cell/enucleated bovine oocyte derived NT units, sufficient cells to produce an ES cell colony, typically on the order of about 50 cells, will be obtained about 12 days after initiation of oocyte activation. However, this may vary dependent upon the particular cell used as the nuclear donor, the species of the particular oocyte, and culturing conditions. One skilled in the an can readily ascertain visually when a desired sufficient number of cells has been obtained based on the morphology of the cultured NT units.

In the case of human/human nuclear transfer embryos, or other embryos produced using non-human primate donor or oocyte, it maybe advantageous to use culture medium known to be useful for maintaining human and other primate cells in tissue culture. Examples of a culture media suitable for human embryo culture include the medium reported in Jones et al., Human Reprod., 13(1): 169-177 (1998), the P1-catalog #99242 medium, and the P-1 catalog #99292 medium, both available from Irvine Scientific. Santa Ana, Calif. and those used by Thomson et al., (1998) and (1995), which references are incorporated by reference in their entirety.

Another preferred medium comprises: ACM, uridine, glucose, 1000 IU of LIF.

As discussed above, the cells used in the present invention will preferably comprise mammalian somatic cells., most preferably cells derived from an actively proliferating (non-quiescent) mammalian cell culture. In an especially preferred embodiment, the donor cell will be genetically modified by the addition, deletion or substitution of a desired DNA sequence. For example, the donor cell. e.g. a keratinocyte or fibroblast. e.g. of human, primate or bovine origin, may be transfected or transformed with a DNA construct that provides for the expression of a desired gene product, e.g., therapeutic polypeptide. Examples thereof include lymphokines, e.g., IGF-I, IGF-II, interferons, colony stimulating factors, connective tissue polypeptides such as collagens, genetic factors, clotting factors, enzymes, enzyme inhibitors. etc.

Also, as discussed above, the donor cells may be modified prior to nuclear transfer, e.g. to effect impaired cell lineage development, enhanced embryonic development and/or inhibition of apoptosis. Examples of desirable modifications are discussed further below.

One aspect of the invention will involve genetic modification of the donor cell. e.g., a human cell, such that it is lineage deficient and therefore hen used for nuclear transfer it will be unable to give rise to a viable offspring. This is desirable especially in the context of human nuclear transfer embryos, wherein for ethical reasons, production of a viable embryo may be an unwanted outcome. This can be effected by genetically engineering a human cell such that it is incapable of differentiating into specific cell lineages when used for nuclear transfer. In particular, cells may be genetically modified such that when used as nuclear transfer donors the resultant “embryos” do not contain or substantially lack at least one of mesoderm, endoderm or ectoderm tissue.

This can be accomplished by. e.g. knocking-out or impairing the expression of one or more mesoderm, endoderm or endoderm specific genes. Examples thereof include:

Mesoderm: SRF, MESP-1, HNF-4, beta-I integrin, MSD;

Endoderm: GATA-6, GATA-4;

Ectoderm: RNA helicase A, H beta 58.

The above list is intended to be exemplar and non-exhaustive of known genes which are involved in the development of mesoderm, endoderm and ectoderm. The generation of mesoderm deficient, endoderm deficient and ectoderm deficient cells and embryos has been previously reported in the literature. See, e.g. Arsenian et al., EMBO J., 17(2): 6289-99 (1998); Saga. Mech. Dev., 75(1-2): 53-66 (1998). Holdener et al. Development. 120(5): 1355-1346 (1994); Chen et al., Genes Dev. 8(20): 2466-77 (1994): Rohwedel et al. Dev. Biol., 201(2): 167-89 (1998) (mesoderm); Morrisey et al. Genes. Dev. 12(22): 3579-90 (1998); Soudais et al. Development, 121(11):3877-88 (1995) (endoderm); and Lee et al., Proc. Natl. Acad. Sci. USA, 95(23): 13709-13 (1998); and Radice et al., Development. 111(3): 801-11 (1991) (ectoderm).

In general, a desired somatic cell. e.g. a human keratinocyte, epithelial cell or fibroblast, will be genetically engineered such that one or more genes specific to particular cell lineages are “knocked-out” and/or the expression of such genes significantly impair ed. This may be effected by known methods, e.g. homologous recombination. A preferred genetic system for effecting “knock-out” of desired genes is disclosed by Capecchi et al., U.S. Pat. Nos. 5,631,153 and 5,464,764, which reports positive-negative selection (PNS) vectors that enable targeted modification of DNA sequences in a desired mammalian genome. Such genetic modification will result in a cell that is incapable of differentiating into a particular cell lineage when used as a nuclear transfer donor.

This genetically modified cell will be used to produce a lineage-defective nuclear transfer embryo. i.e., that does not develop at least one of a functional mesoderm, endoderm or ectoderm. Thereby, the resultant embryos, even if implanted, e.g., into a human uterus, would not give rise to a viable offspring. However, the ES cells that result from such nuclear transfer will still be useful in that they will produce cells of the one or two remaining non-impaired lineage. For example, an ectoderm deficient human nuclear transfer embryo will still give rise to mesoderm and endoderm derived differentiated cells. An ectoderm deficient cell can be produced by deletion and/or impairment of one or both of RNA helicase A or H beta 58 genes.

These lineage deficient donor cells may also be genetically modified to express another desired DNA sequence.

Thus, the genetically modified donor cell will give rise to a lineage-deficient blastocyst which, when plated, will differentiate into at most two of the embryonic germ layers.

Alternatively, the donor cell can be modified such that it is “mortal.” This can be achieved by expressing antisense or ribozyme telomerase genes. This can be effected by known genetic methods that will provide for expression of antisense DNA or ribozymes, or by gene knockout. These “mortal” cells, when used for nuclear transfer, will not be capable of differentiating into viable offspring.

Another preferred embodiment of the present invention is the production of nuclear transfer embryos that grow more efficiently in tissue culture. This is advantageous in that it should reduce the requisite time and necessary fusions to produce ES cells and/or offspring (if the blastocysts are to be implanted into a female surrogate). This is desirable also because it has been observed that blastocysts and ES cells resulting from nuclear transfer may have impaired development potential. While these problems may often be alleviated by alteration of tissue culture conditions, an alternative solution is to enhance embryonic development by enhancing expression of genes involved in embryonic development.

For example, it has been reported that the gene products of the Ped type, which are members of the MHC I family, are of significant importance to embryonic development. More specifically, it has been reported in the case of mouse preimplantation embryos that the Q7 and Q9 genes are responsible for the “fast growth” phenotype. Therefore, it is anticipated that introduction of DNAs that provide for the expression of these and related genes, or their human or other mammalian counterparts into donor cells, will give rise to nuclear transfer embryos that grow more quickly. This is particularly desirable in the context of cross-species nuclear transfer embryos which may develop less efficiently in tissue culture than nuclear transfer embryos produced by fusion of cells or nuclei of the same species.

In particular, a DNA construct containing the Q7 and/or Q9 gene will be introduced into donor somatic cells prior to nuclear transfer. For example, an expression construct can be constructed containing a strong constitutive mammalian promoter operably linked to the Q7 and/or Q9 genes, an IRES, one or more suitable selectable markers, e.g., neomycin. ADA, DHFR, and a poly-A sequence, e.g., bGH polyA sequence. Also, it may be advantageous to further enhance Q7 and Q9 gene expression by the inclusion of insulates. It is anticipated that these genes will be expressed early on in blastocyst development as these genes are highly conserved in different species, e.g., bovines, goats, porcine, dogs cats and humans. Also, it is anticipated that donor cells can be engineered to affect other genes that enhance embryonic development. Thus, these genetically modified donor cells should produce blastocysts and preimplantation stage embryos more efficiently.

Still another aspect of the invention involves the construction of donor cells that are resistant to apoptosis. i.e., programmed cell death. It has been reported in the literature that cell death related genes are present in preimplantation stage embryos (Adams et al., Science, 281(5381): 1322-6 (1995)). Genes reported to induce apoptosis include e.g. Bad. Bok, BH3, Bik, Hrk, BNIP3 Bim_{L}, Bad, Bid, and EGL-1. By contrast, genes that reportedly protect cells from programmed cell death include, by way of example, BcL-XL, Bcl-w, Mcl-1, A1, Nr-13, BHRF-1, LMW5-HL, ORF16, Ks-Bel-2, E1B-19K, and CED-9.

Thus, donor cells can be constructed wherein genes that induce apoptosis are “knocked out” or wherein the expression of genes that protect the cells from apoptosis is enhanced or turned on during embryonic development.

For example, this can be effected by introducing a DNA construct that provides for regulated expression of such protective genes, e.g., Bcl-2 or related genes during embryonic development. Thereby, the gene can be “turned on” by culturing the embryo under specific growth conditions. Alternatively, it can be linked to a constitutive promoter.

More specifically., a DNA construct containing a Bcl-2 gene operably linked to a regulatable or constitutive promoter, e.g. PGK, S140 CMV, ubiquitin, or β-actin, an IRES, a suitable selectable marker, and a poly-A sequence can be constructed and introduced into a desired donor mammalian cell. e.g., human keratinocyte or fibroblast.

These donor cells, when used to produce nuclear transfer embryos, should be resistant to apoptosis and thereby differentiate more efficiently in tissue culture. Thereby, the speed and/or number of suitable preimplantation embryos produced by nuclear transfer can be increased.

Another means of accomplishing the same result is to impair the expression of one or more genes that induce apoptosis. This will be effected by knock-out or by the use of antisense or ribozymes against genes that are expressed in and which induce apoptosis early on in embryonic development. Examples thereof are identified above. Cell death genes that may be expressed in the antisense orientation include BAX. Apaf-1, and capsases. Additionally, a transgene may be introduced that encodes for methylase or demethylase in the sense or antisense orientation. DNAs that encode methylase and demethylase enzymes are well known in the art. Still alternatively, donor cells may be constructed containing both modifications, i.e., impairment of apoptosis-inducing genes and enhanced expression of genes that impede or prevent apoptosis. The construction and selection of genes that affect apoptosis, and cell lines that express such genes, is disclosed in U.S. Pat. No. 5,646,008, which patent is incorporated by reference herein. Many DNAs that promote or inhibit apoptosis have been reported and are the subject of numerous patents.

Another means of enhancing cloning efficiency is to select cells of a particular cell cycle stage as the donor cell. It has been reported that this can have significant effects on nuclear transfer efficiency (Barnes et al., Mol. Reprod. Devel., 36(1): 33-41 (1993)). Different methods for selecting cells of a particular cell cycle stage have been reported and include serum starvation (Campbell et al., Nature 380: 64-66 (1996); Wilmut et al., Nature, 385: 810-3 (1997)), and chemical synchronization (Urbani et al., Exp. Cell Res., 219: 159-68 (1995)). For example, a particular cyclin DNA may be operably linked to a regulatory sequence, together with a delectable marker, e.g., green fluorescent protein (GFP), followed by the cyclin destruction box, and optionally insulation sequences to enhance cyclin and marker protein expression. Thereby, cells of a desired cell cycle can be easily visually detected and selected for use as a nuclear transfer donor. An example thereof is the cyclin D1 gene in order to select for cells that are in G1. However, any cyclin gene should be suitable for use in the claimed invention. (See, e.g., King et al., Mol. Biol. Cell. 7(9): 1343-57 (1996)).

However, a less invasive or more efficient method for producing cells of a desired cell cycle stale are needed. It is anticipated that this can be effected by genetically modifying donor cells such that they express specific cyclins under delectable conditions. Thereby, cells of a specific cell cycle can be readily discerned from other cell cycles.

Cyclins are proteins that are expressed only during specific stages of the cell cycle. They include cyclin D1, D2 and D3 in G1 phase, cyclin B1 and B2 in G2/M phase and cyclin E, A and H in S phase. These proteins are easily translated and destroyed in the cytogolcytosol. This “transient” expression of such proteins is attributable in part to the presence of a “destruction box”, which is a short amino acid sequence that is part of the protein that functions as a tag to direct the prompt destruction of these proteins via the ubiquitin pathway (Adams et al., Science. 281 (5321): 1322-26 (1998)).

In the present invention, donor cells will be constructed that express one or more of such cyclin genes under easily detectable conditions, preferably visualization. e.g. by the use of a fluorescent label. For example, a particular cyclin DNA may be operably linked to a regulatory sequence, together with a detectable marker. e.g. green fluorescent protein (GFP), followed by the cyclin destruction box, and optionally insulation sequences to enhance cyclin and/or marker protein expression. Thereby, cells of a desired cell cycle can be easily visually detected and selected for use as a nuclear transfer donor. An example thereof is the cyclin D1 gene which can be used to select for cells that are in G1. However, any cyclin gene should be suitable for use in the claimed invention. (See, e.g. King et al. Mol. Biol. Cell, 7(9):1343-57 (1996)).

As discussed, the present invention provides different methods for enhancing nuclear transfer efficiency, preferably a cross-species nuclear transfer process. While the present inventors have demonstrated that nuclei or cells of one species when inserted or fused with an enucleated oocyte of a different species can give rise to nuclear transfer embryos that produce blastocysts, which embryos can give rise to ES cell lines, the efficiency of such process is quite low. Therefore, many fusions typically need to be effected to produce a blastocyst the cells of which may be cultured to produce ES cells and ES cell lines. Yet another means for enhancing the development of nuclear transfer embryos in vitro is by optimizing culture conditions. One means of achieving this result will be to culture NT embryos under conditions impede apoptosis. With respect to this embodiment of the invention, it has been found that proteases such as capsases can cause oocyte death by apoptosis similar to other cell types. (See. Jurisicosva et al., Mol. Reprod. Devel. 51(3): 243-53 (1998)).

It is anticipated that blastocyst development will be enhanced by including in culture media used for nuclear transfer and to maintain blastocysts or culture preimplantation stage embryos one or more capsase inhibitors. Such inhibitors include by way of example capsase-4 inhibitor I, capsase-3 inhibitor I, capsase-6 inhibitor II, capsase-9 inhibitor II, and capsase-1 inhibitor I. The amount thereof will be an amount effective to inhibit apoptosis, e.g., 0.00001 to 5.0% by weight of medium, more preferably 0.01% to 1.0% by weight of medium. Thus, the foregoing methods may be used to increase the efficiency of nuclear transfer by enhancing subsequent blastocyst and embryo development in tissue culture.

After NT units of the desired size are obtained, the cells are mechanically removed from the zone and are then used to produce EG. ES or ES-like cells or cell lines. This is preferably effected by taking the clump of cells which comprise the NT unit, which typically will contain at least about 50 cells, washing such cells, and plating the cells onto a feeder layer. e.g., irradiated fibroblast cells. Typically, the cells used to obtain the stem-like cells or cell colonies will be obtained from the inner most portion of the cultured NT unit which is preferably at least 50 cells in size. However. NT units of smaller or greater cell numbers as well as cells from other portions of the NT unit may also be used to obtain ES-like cells and cell colonies.

It is further envisioned that a longer exposure of donor cell DNA to the oocyte's cytosol may facilitate the dedifferentiation process. This can be accomplished by re-cloning, i.e., by taking blastomeres from a reconstructed embryo and fusing them with a new enucleated oocyte. Alternatively, the donor cell may he fused with an enucleated oocyte and four to six hours later, without activation, chromosomes removed and fused with a younger oocyte. Activation would occur thereafter.

The cells are maintained in the feeder layer in a suitable growth medium, e.g. alpha MEM supplemented with 10% FCS and 0.1 mM β-mercaptoethanol (Sigma) and L-glutamine. The growth medium is changed as often as necessary to optimize growth. e.g., about every 2-3 days.

This culturing process results in the formation of embryonic or stem-like cells or cell lines. In the case of human cell/bovine oocyte declined NT embryos, colonies are observed by about the second day of culturing in the alpha MEM medium. However, this time may vary dependent upon the particular nuclear donor cell, specific oocyte and culturing to conditions. One skilled in the an can vary the culturing conditions as desired to optimize growth of the particular embryonic or stem-like cells. Other suitable media are disclosed herein.

Alternatively, that such cells are actual human or primate embryonic stein cells will be confirmed based on their capability of giving rise to all of mesoderm, ectoderm and endoderm tissues. This will be demonstrated by culturing ES cells produced according to the invention under appropriate conditions, e.g., as disclosed by Thomsen, U.S. Pat. No. 5,843,780, incorporated by reference in its entirety herein. Alternatively, the fact that the cells produced according to the invention are pluripotent will be confirmed by injecting such cells into an animal, e.g., a SCID mouse, or large agricultural animal, and thereafter obtaining tissues that result from said implanted cells. These implanted ES cells should give rise to all different types of differentiated tissues, i.e., mesoderm, ectoderm, and endodermal tissues.

The resultant ES. EG. ES-like cells and cell lines have numerous therapeutic and diagnostic applications. For example, such embryonic or stem-like cells may be used for cell transplantation therapies. Human embryonic or stem-like cells have application in the treatment of numerous disease conditions.

Still another object of the present invention is to improve the efficacy of nuclear transfer, e.g., cross-species nuclear transfer by introducing mitochondrial DNA of the same species as the donor cell or nucleus into the recipient oocyte before or after nuclear transfer, before or after activation, and before or after fusion and cleavage. Preferably, if the donor cell is human, human mitochondrial DNA will be derived from cells of the particular donor. e.g., liver cells and tissue.

Methods for isolating mitochondria are well known in the art. Mitochondria can be isolated from cells in tissue culture, or from tissue. The particular cells or tissue will depend upon the particular species of the donor cell. Examples of cells or tissues that may be used as sources of mitochondria include fibroblasts, epithelium, liver, lung, keratinocyte, stomach, heart, bladder, pancreas, esophageal lymphocytes, monocytes, mononuclear cells, cumulus cells, uterine cells, placental cells, intestinal cells, hematopoietic cells, and tissues containing such cells.

For example, mitochondria can be isolated from tissue culture cells and rat liver. It is anticipated that the same or similar procedures may be used to isolate mitochondria from other cells and tissues. As noted above, preferred source of mitochondria comprises human liver tissue because such cells contain a large number of mitochondria. Those skilled in the art will be able to modify the procedure as necessary, dependent upon the particular cell line or tissue. The isolated DNA can also be further purified, if desired, known methods, e.g., density gradient centrifugation.

In this regard, it is known that mouse embryonic stem (ES) cells are capable of differentiating into almost any cell type e.g., hematopoietic stem cells. Therefore, human ES or ES-like cells as well as that of other species produced according to the invention should possess similar differentiation capacity. The ES. EG and EG-like cells according to the invention will be induced to differentiate to obtain the desired cell types according to known methods. For example, the subject ES. EG and EG-like cells may be induced to differentiate into hematopoietic stem cells, muscle cells, cardiac muscle cells, liver cells, cartilage cells, epithelia) cells, urinary tract cells, etc. by culturing such cells in differentiation medium and under conditions which provide for cell differentiation. Medium and methods which result in the differentiation of embryonic stem cells are known in the art as are suitable culturing conditions.

For example, Palacios et al., Proc. Natl. Acad. Sci. USA, 92: 7530-37 (1995) teaches the production of hematopoietic stem cells from an embryonic cell line by subjecting stem cells to an induction procedure comprising initially culturing aggregates of such cells in a suspension culture medium lacking retinoic acid followed by culturing in the same medium containing retinoic acid, followed by transferral of cell aggregates to a substrate which provides for cell attachment.

Moreover, Pedersen, J. Reprod. Fertil. Dev., 6: 543-52 (1994) is a review article which references numerous articles disclosing methods for in vitro differentiation of embryonic stem cells to produce various differentiated cell types including hematopoietic cells, muscle, cardiac muscle, nerve cells, among others.

Further, Bain et al., Dev. Biol., 168:342-357 (1995) leaches in vitro differentiation of embryonic stem cells to produce neural cells which possess neuronal properties. These references are exemplar of reported methods for obtaining differentiated cells from embryonic or stem-like cells. These references and in particular the disclosures therein relating to methods for differentiating embryonic stem cells are incorporated by reference in their entirety herein.

Thus, using known methods and culture medium, one skilled in the an may culture the subject embryonic or stem-like cells to obtain desired differentiated cell types, e.g. neural cells, muscle cells, hematopoietic cells, etc. In addition, the use of inducible Bcl-2 or Bcl-x1 might be useful for enhancing in vitro development of specific cell lineages. In vivo, Bcl-2 prevents many, but not all, forms of apoptotic cell death that occur during lymphoid and neural development. A thorough discussion of how Bcl-2 expression might be used to inhibit apoptosis of relevant cell lineages following transfection of donor cells is disclosed in U.S. Pat. No. 5,646,008, which is herein incorporated by reference.

The subject embryonic or stem-like cells may be used to obtain any desired differentiated cell type. Therapeutic usages of such differentiated human cells are unparalleled. For example, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Hematopoietic stem cells can be obtained, e.g., by fusing adult somatic cells of a cancer or AIDS patient. e.g., epithelial cells or lymphocytes with an enucleated oocyte, e.g., bovine oocyte, obtaining embryonic or stem-like cells as described above, and culturing such cells under conditions which favor differentiation, until hematopoietic stem cells are obtained. Such hematopoietic cells may be used in the treatment of diseases including cancer and AIDS.

Alternatively, adult somatic cells from a patient with a neurological disorder may be fused with an enucleated animal oocyte, e.g., a primate or bovine oocyte, human embryonic or stem-like cells obtained therefrom, and such cells cultured under differentiation conditions to produce neural cell lines. Specific diseases treatable by transplantation of such human neural cells include, by way of example, Parkinson's disease. Alzheimer's disease, ALS and cerebral palsy, among others. In the specific case of Parkinson's disease, it has been demonstrated that transplanted fetal brain neural cells make the proper connections with surrounding cells and produce dopamine. This can result in long-term reversal of Parkinson's disease symptoms.

To allow for specific selection of differentiated cells, donor cells may be transfected with selectable markers expressed via inducible promoters, thereby permitting selection or enrichment of particular cell lineages when differentiation is induced. For example, CD34-neo may be used for selection of hematopoietic cells, Pwl-neo for muscle cells, Mash-1-neo for sympathetic neurons, Mal-neo for human CNS neurons of the grey matter of the cerebral cortex. etc.

The great advantage of the subject invention is that it provides an essentially limitless supply of isogenic or synegenic human cells suitable for transplantation. Therefore, it will obviate the significant problem associated with current transplantation methods, i.e., rejection of the transplanted tissue which may occur because of host versus graft or graft versus host rejection. Conventionally, rejection is prevented or reduced by the administration of anti-rejection drugs such as cyclosporin. However, such drugs have significant adverse side-effects. e.g., immunosuppression, carcinogenic properties, as well as being very expensive. The present invention should eliminate, or at least greatly reduce, the need for anti-rejection drugs, such as cyclosporine, imulan, FK-506, glucocorticoids, and rapamycin, and derivatives thereof.

Other diseases and conditions treatable by isogenic cell therapy include, by way of example, spinal cord injuries, multiple sclerosis, muscular dystrophy, diabetes, liver diseases, i.e., hypercholesterolemia, heart diseases, cartilage replacement, burns, foot ulcers, gastrointestinal diseases, vascular diseases, kidney disease, urinary tract disease, and aging related diseases and conditions.

Also, human embryonic or stem-like cells produced according to the invention may be used to produce genetically engineered or transgenic human differentiated cells. Essentially, this will be effected by introducing a desired gene or genes, which may be heterologous or removing all or pail of an endogenous gene or genes of human embryonic or stem-like cells produced according to the invention, and allowing such cells to differentiate into the desired cell type. A preferred method for achieving such modification is by homologous recombination because such technique can be used to insert, delete or modify a gene or genes at a specific site or sites in the stem-like cell genome.

This methodology can be used to replace defective genes. e.g. defective immune system genes, cystic fibrosis genes, or to introduce genes which result in the expression of therapeutically beneficial proteins such as growth factors, lymphokines, cytokines, enzymes etc. For example, the gene encoding brain derived growth factor may be introduced into human embryonic or stem-like cells, the cells differentiated into neural cells and the cells transplanted into a Parkinson's patient to retard the loss of neural cells during such disease.

Previously, cell types transfected with BDNF varied from primary cells to immortalized cell lines, either neural or non-neural (myoblast and fibroblast) derived cells. For example, astrocytes have been transfected with BDNF gene using retroviral vectors, and the cells grafted into a rat model of Parkinson's disease (Yoshimoto et al., Brain Research, 691:25-36, (1995)).

This ex vivo therapy reduced Parkinson's-like symptoms in the rats up to 45% 32 days after transfer. Also, the tyrosine hydroxylase gene has been placed into astrocytes with similar results (Lundberg et al., Develop. Neurol., 139:39-53 (1996) and references cited therein).

However, such ex vivo systems have problems. In particular, retroviral vectors currently used are down-regulated in vivo and the transgene is only transiently expressed (review by Mulligan, Science, 260: 926-932 (1993)). Also, such studies used primary cells, astrocytes, which have finite life span and replicate slowly. Such properties adversely affect the rate of transfection and impede selection of stably transfected cells. Moreover, it is almost impossible to propagate a large population of gene targeted primary cells to be used in homologous recombination techniques.

By contrast, the difficulties associated with retroviral systems should be eliminated by the use of human ES and ES-like cells and cell lines. It has been demonstrated previously by the subject assignee that cattle and pig embryonic cell lines can be transfected and selected for stable integration of heterologous DNA. Such methods are described in commonly assigned U.S. Ser. No. 08/626,054, filed Apr. 1, 1996, now U.S. Pat. No. 5,905,042, incorporated by reference in its entirety. Therefore, using such methods or other known methods, desired genes may be introduced into the subject ES and ES-like cells, and the cells differentiated into desired cell types, e.g., hematopoietic cells, neural cells, pancreatic cells, cartilage cells. etc.

Genes which maybe introduced into the subject EG, ES. ES-like cells include, by way of example, epidermal growth factor, basic fibroblast growth factor, glial derived neurotrophic growth factor, insulin-like growth factor (I and II), neurotrophin-3, neurotrophin-4/5, ciliary neurotrophic factor, AFT-1, cytokine genes (interleukins, interferons, colony stimulating factors, tumor necrosis factors (alpha and beta), etc.), genes encoding therapeutic enzymes, collagen, human serum albumin, etc.

In addition, it is also possible to use one of the negative selection systems now known in the art for eliminating therapeutic cells from a patient if necessary. For example, donor cells transfected with the thymidine kinase (TK) gene will lead to the production of embryonic cells containing the TK gene. Differentiation of these cells will lead to the isolation of therapeutic cells of interest which also express the TK gene. Such cells may be selectively eliminated at any time from a patient upon gancyclovir administration. Such a negative selection system is described in U.S. Pat. No. 5,698,446, and is herein incorporated by reference.

The subject ES, ES-like and EC cells may be used as an in vitro model of differentiation, in particular for the study of genes which are involved in the regulation of early development.

Also, differentiated cell tissues and organs using the subject embryonic or stem-like cells may be used in drug studies.

Further, the subject cells may be used to express recombinant DNAs.

Still further, the subject embryonic or stem-like cells may be used as nuclear donors for the production of other embryonic or stem-like cells and cell colonies.

Also, cultured inner cell mass, or stem cells, produced according to the invention may be introduced into animals. e.g., SCID mice, cows, pigs, e.g., under the renal capsule or intramuscular and used to produce a teratoma therein. This teratoma can be used to derive different tissue types. Also, the inner cell mass produced by X-species nuclear transfer may be introduced together with a biodegradable, biocompatible polymer matrix that provides for the formation of 3-dimensional tissues. After tissue formation, the polymer degrades, ideally just leaving the donor tissue, e.g., cardiac, pancreatic, neural, lung, liver. In some instances, it may be advantageous to include growth factors and proteins that promote angiogenesis. Alternatively, the formation of tissues can be effected totally in vitro, with appropriate culture media and conditions, growth factors, and biodegradable polymer matrices.

### EXAMPLES

### Example 1

We have developed a method, using nuclear transplantation, to produce transgenic embryonic stem (ES)-like cells from fetal bovine fibroblasts. These cells, when reintroduced into preimplantation embryos, differentiated into derivatives from the three embryonic germ layers, ectoderm, mesoderm, and endoderm, in 5-month-old animals. Six out of seven (86%) calves born were found to be chimeric for at least one tissue. These experiments demonstrate that somatic cells can be genetically modified and then de-differentiated by nuclear transfer into ES-like cells, opening the possibility of using them in differentiation studies and human cell therapy.

Embryonic stem (ES) cells have been available for several strains of mice for many years and have been shown to be capable of contributing to each of the tissues of the animal when combined with a host embryo to form a chimera. Techniques have been developed for inducing the differentiation of mouses ES cells in vitro and successfully transplanting them into recipient mice. Success in developing pluripotent cell lines from large animal species, such as bovine, has been minimal. Production of putative bovine ES cells vas first reported by Saito et al., and later, a similar type of stem-like cells w as reported to direct development through organogenesis. Bovine ES cells that are capable of complete differentiation to term, in vivo, have not been reported. Little success has been achieved in inducing ES cells to differentiate into a specified tissue in vitro or in the selecting specific cells, out of the many other types of cells that are present, following the induction of in vitro differention.

The objectives of this study were to develop an efficient procedure for producing bovine ES-like cells, to lest the pluripotency of these cells in vivo by forming chimeras with host embryos, and to develop an efficient method for genetic modification of the cells using somatic cell nuclear transplantation.

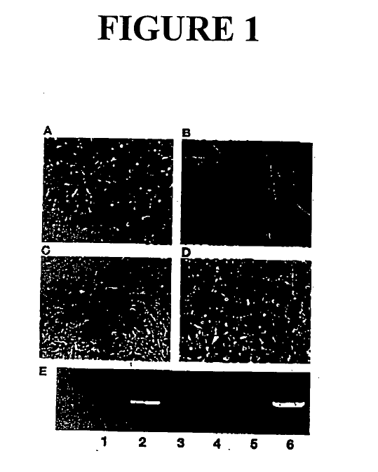

Results. Production of transgenic embryo-derived pluripotent ES-like cell colonies. As one approach to producing transgenic cattle, putative bovine ES-like cells were derived from embryos. In vitro maturation and fertilization of oocytes and in vitro culture of the embryos to the blastocyst stage produced 49 embryos at day 7. Blastocysts were mechanically dissected and plated on mitotically inactivated fetal mouse fibroblast feeder layers. Twenty-seven inner cell masses attached to the feeder layer grew as ES-like cell colonies and successfully survived passaging over at least 12 months without differentiation. These colonies had well-defined edges. Cells in these colonies had a high nuclear to cytoplasmic ratio and a high density of cytoplasmic lipid granules, and were negative for cytokeratin and vimentin. Unlike mouse ES cells, bovine ES cells eventually formed single layer sheets (FIG. 1A) and were alkaline phosphatase negative.

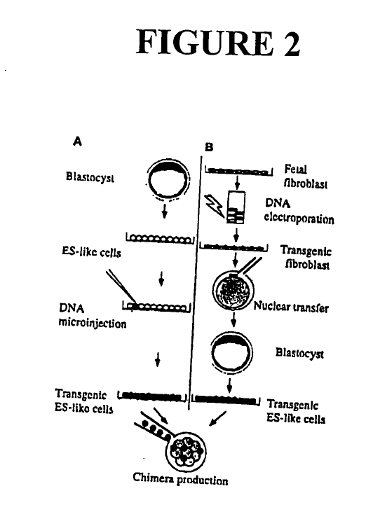

The method of producing transgenic bovine ES-like cells also differed from procedures used for the mouse (FIG. 2A). Bovine ES-like cells, unlike mouse ES cells, do not survive replating when trypsinization is performed; therefore, mechanical passage was used instead. Passage of the cells mechanically involves removing a group of cells, containing a minimum of 50 to 100 cells, and transferring these to fresh feeder layers. Because single cell suspensions could not be passaged, it was not possible to use electroporation for DNA transfection or to clonally propagate transgenic cells. Therefore microinjection of DNA into the nucleus of individual cells was used as an alternative method. Approximately 500 to 1000 cells could be injected per hour, and injection volume vas based on nuclear swelling. Three different cell lines ere used. A cytomegalovirus (CMV)-β-galactosidase-neomycin (β-Geo) cassette as delivered into the nucleus of ES-like cells. Five, three, and zero stable. G418 selected transgenic colonies were produced out of 3753, 3508S and 3502 injected cells, respectively. We did not determine if these colonies were derived from single or multiple transgenic cells. During G418 selection the original colony essentially disappeared before growth of the transgenic cells began, indicating a possible clonal origin; however, the possibility of having produced a transgenic colony from two or more closely placed transgenic cells cannot be ruled out. β-galactosidase expression as consistently high in all colonies, although not all cells within a colony expressed the gene (FIG. 1B). PCR amplification of a segment of the transgene also confirmed that the cells were transgenic (FIG. 1E).

Production of transgenic somatic cell-derived bovine pluripotent ES-like cell colonies. Although transgenic ES-like cells can be produced by microinjection, the generation of a large number of transgenic ES-like cells and clonal propagation was not achieved. Therefore, we took another approach (FIG. 2B) that involved transfection of bovine fetal fibroblasts and fusion of the transgenic fibroblast cells to enucleated oocytes to produce blastocyst stage nuclear transplant embryos. These embryos were then plated on fibroblast feeder layers to produce transgenic ES-like cell colonies. Bovine fibroblasts were obtained from 55 day fetus, and grown and transfected by electroporation using standard methods (FIG. 1C). Three hundred and thirty enucleated mature bovine oocytes were reconstructed with actively dividing fibroblasts. Thirty-seven (11%) blastocytes (day 7.5) were obtained and ES-like cell lines were established from 22 (59%) of these. Out of 22 cell lines, 21 were positive for the transgene after PCR amplification of the β-galactosidase fragment. The negative ES-like colony could have originated from a neomycin-resistant fibroblast that lost the β-galactosidase gene. Fibroblast-derived ES-like cell colonies showed morphology and cytoplasmic marker characteristics identical to those of embryo-derived ES-like cells (FIG. 1D). Furthermore, colonies were passaged for several months without differentiation, even, in one case, when a colony as derived from a senescent, nondividing fibroblast cell line.

Production of chimeric calves. In order to determine the potency of bovine embryo- (passage 10) and fibroblast-derived ES-like cells (passage 3) in vivo. 8 to 10 cells were introduced into day 3 in vitro produced embryos, cultured in vitro until day 7.5 and transferred into synchronized recipients. Five calves were born from embryos that received transgenic embryo-derived ES-like cells, and seven calves ere born from embryos that received transgenic nuclear-transfer (NT)-derived ES-like cells (Table 1). All the animals were phenotypically normal.

TABLE 1
Production of transgenic calves using embryo,
and NT-derived ES like cells

| | Injected | Blastocyst | Blastocyst | Calves | Transgenic |
|---|---|---|---|---|---|
| | embryos | produced (%) | transferred | born | calves* |
| Embryo ES-like cells | 70 | 16 (23) | 16 | 5 | 3 |
| NT ES-like cells | 99 | 22 (22) | 10 | 7 | 6 |

*Animals with at least one transgenic tissue.

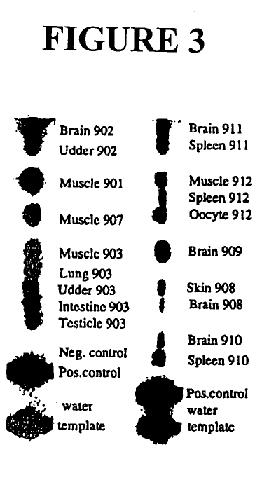

All the animals were slaughtered at 5 months of age, with the exemption of calf 904, which was killed at 45 days of age. Genomic DNA was isolated from a spectrum of tissues (skin, muscle, brain, liver, spleen, kidney, heart, lung, mammary gland, intestine, and gonads) from each animal, amplified using β-Geo primers, and probed using standard-protocol Southern blot analysis. Results were positive in at least one tissue in nine calves and in two or more tissues in six calves. Oocytes were found to be positive in one animal (FIG. 3). The limited presence of transgenic cells in the newborn animals could be attributed to the fact that not all the ES-like cells were incorporated into the developing morulas; moreover, among those cells that did incorporate, degree of pluripotency may have varied.

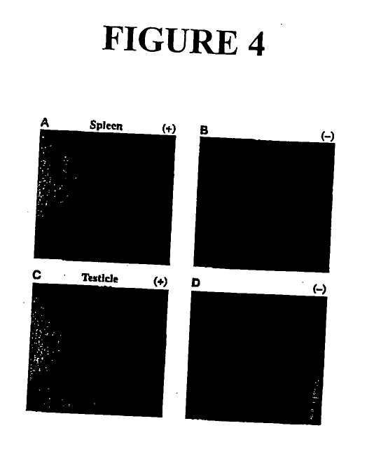

Fluorescent in situ hybridization (FISH) analysis was performed in spleen tissue from calf 911 (FIG. 4A), and testis of calf 903 (FIG. 1C). Positive hybridization signals were identified in both tissues. In the spleen. 32% of nuclei (82/256) exhibited green signals compared with negative spleen in which only 1% of nuclei (2/231) were classified as carrying green signals. Testis specimens were not presented as a single monolayer of cells: therefore, percentage of positive cells was not assessed; however, positive signals were detected inside the seminiferous tubules.

Discussion. The first objective of this study was to produce bovine pluripotent ES-like cells. ES-like cells are derived from an early stage embryo of the inner cell mass (ICM) directly, and, therefore, should retain the morphology and cellular characteristics of the ICM. In the mouse. ES cells grow as colonies with a defined margin, and cells have high nuclear to cytoplasmic ratio and high density of lipid inclusions similar to the ICM. Our bovine cells derived both from embryos and NT fibroblasts, also retained these characteristics. The expression of various cytoplasmic markers has also been used to indicate an ICM-like quality of mouse ES cells. In the bovine, ES-like cells derived either from embryonic or somatic cell sources, do not express differentiation markers such as vimentin and cytokeratin in a pattern similar to the ICM; however, these cells are alkaline phosphatase negative. The second characteristic of a pluripotent embryonic cell is that it can be grown over many passages without showing signs of differentiation. In this study, and other preliminary work (1), bovine ICM-derived cells were passaged for over 1 year without losing the morphological and cellular similarities to the ICM. The third and most important characteristic used to define ES-like cells is that, upon introduction into a preimplantation) embryo, they are able to colonize the ectodermal, mesodermal, and endodermal tissues and the germ line, as the host embryo develops and differentiates. In this study it was shown that both embryonic and fibroblast-derived ES-like bovine ells are capable of giving rise to multiple tissues in 5-month-old animals. Our results demonstrate that cells derived from somatic and embryonic sources possess functional and phenotypic characteristics of pluripotent ES-like cells.

Much work has been done in many different species toward developing methods of producing ES cells; how ever, little success has been reported at meeting all the criteria listed above. In rabbit (6) production of chimeric offspring was reported, but no chimerism in gonads was demonstrated. In hamster (7, 8) and cow (3, 4), cells were grown in vitro however, no chimeric animals were produced. This is the first published report demonstrating transgenic chimerism in full-term live mammals, including in gonadal tissue from a species other than a mouse. However, until germline transmission is demonstrated, we refer to our cells as “pluripotent or ES-like cells” instead of ES cells.

The results in this study indicate that, although genetic modifications could be made in bovine ES-like cells by microinjection, and transgenic cells could be selected by a standard neomycin resistance approach, limitations in the number of cells that can be microinjected, the slow growth of the cells, and our inability to clonally propagate the ES-like cells limits the usefulness of this approach, particularly for gene targeting. This is one important difference between bovine ES-like cells and mouse ES cells. Aside from the fact that care must be taken to prevent differentiation, mouse ES cells can be readily grown in culture, clonally propagated, transfected by standard high-volume gene transfer methods, and in many cases, exhibit high-efficiency homologous recombination. In our system, the low transfection efficiency of bovine ES-like cells prevents the possibility of using direct ES-like cell transfection for gene targeting.

An alternative method of making genetic modifications in bovine ES-like cells is to genetically modify fibroblast cells and then produce embryos by nuclear transplantation. Genetic modification is relatively simple with fibroblast cells, which are easy to grow, transfect and clonally propagate. Furthermore, gene targeting and selection for homozygous lines in vitro have been successful in human fibroblast lines.

This study demonstrates that ES-like cells can be produced from bovine embryos, which can be cultured without a change in morphology for indefinite periods in vitro and retain the ability to give rise to tissues derived from all three gene layers in offspring. Furthermore, using nuclear transplantation., these cells can be produced from genetically modified fibroblasts. This system could be useful for the in vitro production of genetically modified bovine cells to be used for cell transplant therapies for many different human diseases.

Experimental Protocol

In vitro maturation of bovine oocytes. Ovaries were recovered at a slaughterhouse, placed in warm phosphate-buffered saline (PBS) (34° C.) and brought to the laboratory within a limit of 8 h. Each follicle of more than 2 mm in diameter was aseptically aspirated with an 18 gauge needle. Search of oocytes was performed in modified Tyrode's medium (TL Hepes). Oocytes with a homogeneous cytoplasm, considerable perivitelline space and intact cumulus cells were placed in maturation medium M199 (GIBCO, Grand Island, N.Y.), a 10% fetal calf serum (FCS), 5 μl/ml bovine follicle-stimulating hormone (Nobl, Sioux Center, Iowa), 5 μl/ml bovine luteinizing hormone (Nobl), and 10 μl/ml Pen-strep (Sigma, St. Louis, Mo.) for 22 h at 38.5° C. and 5% CO₂.

In vitro fertilization of bovine oocytes. Twenty-two hours post-maturation, oocytes were placed in fertilization medium (5 ml CR2-Specialty medium, stock solution 100 U/ml penicillin, 100 μg/ml streptomycin, 0.005 μg/ml phenol red, 30 mg bovine serum albumin fatty acid free, 5 μg/ml sodium heparin). A unit of frozen semen was thawed and placed on top of a Percoll layer that contains 90% Percoll (Sigma) and one part 10 modified sperm TL plus, 45% Percoll (one part of 90% Percoll stock solution and one part sperm TI, without BSA). Dead sperm were separated from live sperm by centrifugation at 700 G for 30 min. Sperm pellet was resuspended at a final concentration of 500,000 sperm/ml. After 12 h in culture at 38.5° C. and 5% CO₂, eggs were removed and placed in CR2 medium with 3 mg/ml BSA.

Embryo culture. During the first 3 days after fertilization, embryos were cultured in 500 μl well plates with mouse embryonic fibroblast (MF) feeder layers and CR2 with 6 mg/ml BSA. On day 4, embryos were transferred to 500 μl well plates with MF feeder layers, CR2 with 6 mg/ml BSA, and 10% FCS until blastocyst stage day 7 post-insemination).

ES-like cell culture. Blastocysts were placed in a 32 mm plate (Nunc, Rochester, N.Y.) with mitotically inactivated MF feeder layer and ES medium (Alpha MEM, 10% fetal calf serum, 4 111/ml antibiotic-antimycotic, 2.8 μl/ml 2-mercaptoethanol, 0.3 mg/ml L-glutamine, and 1 μl/ml tylosin tartrate) equilibrated a day in advance at 38.5° C. and 5% CO₂. Using a 22 gauge needle, blastocysts' zona pellucida and trophoblast were mechanically removed. The remaining ICM was placed underneath the MF. After 1 week in culture, ES-like cells were passaged to a fresh mitotically inactivated MF. Inactivation of MF was performed by exposing them to gamma radiation (2956 rads). ES-like cells were passaged by cutting a small piece (50 to 100 cells) of the colony and placed on top MF feeder layers using a pulled Pasteur pipette.

Nuclear transplantation. Eighteen hours post-maturation, oocytes were placed in a 100 μl drop of TL HECM-Hepes under mineral oil (Sigma). Oocyte enucleation (extraction of chromosomes) was performed using a beveled glass pipette of 25 μm diameter. Evaluation of enucleation was done by exposure of individual oocytes previously cultured for 15 min in 1 μg/ml of bisBENZIMIDE (Hoechst 33342; Sigma) in TL HECM-Hepes under ultraviolet light. Donor cells were placed in the perivitelline space and fused with the egg's cytoplasm at 23 h post-maturation. Oocytes and donor cell were placed into 4 ml medium made of 50% SOR2 fusion medium (0.25 M D-sorbitol (Sigma), 100 (M CaOAc (Sigma), 0.5 mM magnesium acetate (Sigma), 1.0 g BSA (Sigma), and 50% HECM-Hepes for 2 min. Eggs were then placed between the electrodes of a 500 μm fusion chamber. Once the eggs were aligned, a pulse of 90 V was administered over 15 μs. Eggs were then returned to the 50/50 medium of SOR2 and HECM/Hepes for 2 min and, finally, placed into a 500 μl drop of CR2 at 38.5° C. and 5% CO₂ until activation.

Oocyte activation. Activation was performed in general as described by Forrester et al., Proc. Natl, acad. Sci. USA 88: 7514-77 (1991) and Palaclos et al., Dev. Biol., 92: 7530-4 (1995). Briefly, 25 to 27 h post-maturation oocytes were incubated in 5 μm ionomycin (Cal Biochem, La Jolla, Calif.), and 2 mM of 6-dimethylaminopurine (DMAP; Sigma) in CR2 with 3 mg/ml of BSA (fatty acid free; Sigma). After activation, eggs were washed in HECM/Hepes five times and placed for culture in a 500 μl well of MF and CR2 with 3 mg/ml of BSA (fatty acid free) at 38.5° C. and 5% CO₂.

Transgenic ES-like cell production. Five micrograms per milliliter of a β-Geo cassette gene were microinjected into the nuclei of bovine ES-like cells. Twenty four to forth-eight hours after microinjection, 150 μg/ml of G418 was added to the culture medium. After 3 weeks under selection, a colony was considered transgenic upon DNA screening by PCR and ethidium bromide gel, and by β-galactosidase staining.

Bovine fibroblast production and electroporation. Bovine fibroblasts were produced from a 55-day-old fetus as follows. Under sterile conditions, the livers, intestines, and beads of the fetuses were discarded. The remaining parts of the fetuses were carefully minced and placed in a solution of Dulbecco's phosphate buffered saline (dpbs) with 0.08% trypsin (Difco, Detroit, Mich.) and 0.02% EDTA (Sigma). After 30 min incubation at 37° C. the supernatant was discarded and the pellet resuspended with trypsin-EDTA/dPBS. After 30 min incubation, the supernatant was removed and centrifuged at 300 G for 10 min. The pellet of cells was then resuspended with ES culture medium and plated in polystyrene tissue culture dishes (25010; Corning, Charlotte, N.C.). After two passages, cells were electroporated with a β-Geo cassette gene with the protocol described by Invitrogen (San Diego, Calif.) for COS cells (11). After 3 weeks under 400 μg per ml of G418 selection, fibroblasts were considered transgenic upon DNA screening by PCR and ethidium bromide gel, and by β-galactosidase staining.

Alkaline phosphatase staining. Culture medium was removed from the plates and cells were fixed with 4% paraformaldehyde for 20 min. Cells were washed three times in Tris-maleate buffer (3.6 g Trizma base [Sigma], in 1 L water, pH raised to 9.0 with 1 M maleic acid) for 10 min each wash. The last wash was removed and the staining solution (Tris-maleate buffer, 200 μl of a 0.5 mM MgCl₂, naphthol A5-MX phosphate [Sigma]), 0.4 mg/ml, Fast blue [Sigma], 1 mg/ml) was added to the cells for 15 to 20 min. Once blue cells were detected, the reaction was stopped by adding PBS which brought the pH to 7.4

Chimera production. Seventy-two hours after in vitro fertilization (eight cell stage), embryos were placed in manipulation medium (HECM/Hepes with 10% FCS and 7.5 μg/ml of cytochalasin B [Sigma]). ES-like cells were dissociated using 0.08% trypsin (Difco) and 0.02% EDTA in PBS during 25 to 30 min. Using a 15-20 μl/ml diameter beveled pipette, 8 to 10 cells were introduced into the embryos. Embryos were placed in a 500 μl culture drop (MF feeder layer, CR2 with 6 mg/ml of BSA and 10% FCS).

Immunohistochemical studies. Primary antibodies specific against cytokeratin 8-18 (Sigma) and vimentin (Sigma) were used in ES-like cell cultures. Cells were plated on sterile glass slides, fixed in 2% paraformaldehyde, and extracted with cold (−20° C.) acetone. Cells were incubated with primary antibody dilutions in PBS containing 0.5% BSA (PBSA) for 1 h at room temperature. Slides were then rinsed three times in PBSA with changes of rinse solution every 10 min, and incubated for 1 h in fluorescein 5-isothiocyanate (FITC) conjugated anti-mouse IgG (Sigma). After rinsing in PBSA for 30 min, cover slips were mounted in 50% glycerol and observed under a fluorescence microscope (8).

β-galactosidase staining. Culture medium was removed from the plates, and cells were fixed with 2% glutaraldehyde in PBS. Then cells were washed three times with PBS and color substrate (5 mM K₃Fe(CN)₆, 5 mM K₄Fe(CN)₆, 1 mM MgCl₂, 1 mg/ml X-gal in PBS, pH 7.0-7.5) was added for 3 h (12).

PCR analysis and blot analysis. Analysis of transfected cells and tissue from 5-month-old animals was performed using a sense primer (ACT3βGeo, a 21 base CGCTGTGGTACACGCTGTGCG) and antisense primer (ACT4βGEO, 1 22 base CACCATCCAGTGCAGGAGCTCG [Amilof Biotech, Boston, Mass.). Reactions were run for 35 cycles (1) heated at 95° C. for 30 s (2) primers were annealed at 65° C. for 1 min, (3) extended for 2 min at 72° C., followed by 10 min extension at 72° C. The amplified product was a 782 bp fragment. Sample analysis was performed by separating by size in a (1%) TAE agarose gel electrophoresis containing ethidium bromide. Products were sized by comparison with markers consisting of 1444 bp, 943 bp, 754 bp, 585 bp, 458 bp, 341 bp, 258 bp, 153 bp, and 105 bp. DNA was then handled according to standard Southern blot analysis protocols. Briefly, DNA was transferred to Zetabind (Cuno, Meriden, Conn.) by capillary transfer and probed with a gel-purified 289 bp ClaI to EcoRV fragment labeled with “PdCTP using random primed labeling kit (Boehringer Mannheim, Indianapolis, Ind.). Hybridization was done at 42° C. overnight. After washing, the blot was exposed to Biomax film (Kodak, Rochester, N.Y.) overnight. Nontransgenic fibroblasts and water were used as negative controls, and transgenic cells for β-Geo and template were used as positive control. When oocytes were analyzed, ovarian follicles were aspirated with a syringe using an 18 gauge needle. Eggs' granulose cells were removed by vortexing the oocyte/cumulus cell complex in 5 mg/ml of hyaluronidase (Sigma) in PBS. Oocytes were washed five times in PBS before DNA isolation.

FISH analysis. Samples were frozen and made onto slides either by slightly pressing the sample against the slide (for spleen slides) or by cryosections (for testis slides), βGeo DNA was linearized with ScaI and biotin-labeled by nick translation reaction. An aliquot of the biotin-labeled DNA was run on a gel and transferred to a membrane, and a streptavidin-alkaline phosphatase assay was performed to detect the size of labeled fragments and quantity of biotin incorporation. The labeled DNA was then co-precipitated with salmon sperm DNA as carrier. A number of single-target single-color FISH assays were performed using varied concentrations of labeled DNA as a probe (250-500 ng). The specimens were washed in 70% acetic acid and digested in pepsin (0.01% in 0.01 M HCl at 37° C.) before denaturation. Testis slides were incubated in pepsin at room temperature for 10 min before warming to 37° C. Denaturation was performed at 75° C. for both chromosomal and probe DNAs and hybridization was allowed to occur for approximately 60 h. Post-hybridization washes included three 5 min washes in 50% formamide/2×SSC and three 5 min washes in 2×SSC at 43° C. Immunochemical detection was achieved with consecutive incubations in FITC-avidin, biotinylated anti-avidin and FITC avidin (Vector, Burlingame, Vt.). Chromatin was counter-stained with DAPI (0.01 μg/ml on antifade; Boehringer Mannheim). After hybridization, slides were coded and blindly analyzed. Analysis was performed in an Olympus BX-60 fluorescence microscope using interference filter sets for single band (DAPI and FITC) and triple band (DAPI, FITC, Texas red). Gray images were acquired using a CCD Camera (Photometrics, Phoenix, Ariz.) and combined using the Oncor (Gaithersburg, Md.) image software.

### REFERENCES

(1) Forrester et al., Proc. Natl. Acad. Sci. USA 88:7514-7577 (1991).

(2) Palaclos et al., Dev. Biol., 92:7530-7534 (1995).

(3) Saito et al., Roux's Arch. Dev. Biol., 201: 134-141 (1992).

(4) Slice et al., Biol. Reprod. 54: 100-110 (1996).

(5) Cibelli et al., Theriogonology 47: 241 (1997).

(6) Schoonjans et al., Mol. Reprod. Dev., 45: 439-443 (1996).

(7) Doelschman et al., Dev. Biol., 127: 224-227 (1988).

(8). Piedrahita et al., Theriogenology, 34: 879-901 (1990).

(9) Brown et al., Science, 277: 831-834 (1997).

(10) Susko-Parrish et al., Dev. Biol., 166: 729-739 (1994).

(11) Invitrogen, The Electroporator manual, Version 3, San Diego, Calif.

(12) Hill et al., Methods Enzymol., 225: 664-681 (1993).

Although the present invention has been described in detail with reference to examples above, it is understood that various modifications can be made without departing from the spirit of the invention, and would be readily known to the skilled artisan. All cited patents and publications referred to in this application are herein incorporated by reference in their entirety.

What is claimed is:

1. An animal produced from embryonic stem cells, where such embryonic stem cells original from a cloned embryo.

2. Poultry produced from embryonic stem cells, where such embryonic stem cells original from a cloned embryo.

3. Mammals produced from embryonic stem cells, where such embryonic stem cells original from a cloned embryo.

4. Farm animals produced from embryonic stem cells, where such embryonic stem cells originate from a cloned embryo and are genetically modified.

5. ES cells produced from an embryo made by nuclear transfer.

6. A business model whereby cryopreserved clonal ES cells are marketed instead of live animals for the production of farm animals.

7. A method for producing an embryonic stem (ES) derived cloned mammal comprising the following steps:
(i) isolating a somatic cell from an animal having desired characteristic(s);
(ii) transfecting such cell with a marker that allows for cells containing to be selected by positive selection;
(iii) using said transfected cell as a cell or nuclear donor during a nuclear procedure;
(iv) culturing the resultant nuclear transfer embryo under conditions that result into development into a blastocyst or post-blastocyst stage embryo;
(v) isolating totipotent (e.g., inner cell mass cells) from said embryo and expanding said cells in culture to produce ES cells;
(vi) optionally cryopreserving said expanded ES cells;
(vii) inserting said ES cells into a host embryo of 1 to 200 cells which is not resistant to the selectable marker;
(viii) culturing the resultant embryo under selective conditions for the selectable marker to obtain embryos that substantially consist of cells that comprise genome of ES cells; and
(ix) after embryos have reached desired size transferring said embryo to a recipient female.

8. The method of claim 7, wherein about 2 to 20 ES cells are inserted into said host embryo.

9. The method of claim 7, wherein the host embryo is a bovine, primate, ovine, murine, porcine, canine, feline, or caprine.

10. A method for deriving a cloned animal from an ES cell comprising:
(i) isolating a somatic cell from an animal having desired characteristics;
(ii) using said cell as a cell or nuclear donor during nuclear transfer;
(iii) using the resultant nuclear transfer fusion to produce an embryo of the blastocyst stage or later;
(iv) isolating totipotent cells (e.g., inner cell mass cells) from said embryo and expanding said cells in culture to produce ES cells;
(v) optionally cryopreserving said ES cells;
(vi) inserting some of said ES cells into a host embryo of 2 to 200 cells which is incapable of development;
(vii) culturing the resultant embryo until it is of a size suitable for implantation into a recipient female;
(viii) transferring said cultured embryo into a recipient female.

11. The method of claim 10, wherein said host embryo is tetraploid.

12. The method of claim 10, wherein the somatic cell is derived from a bovine, porcine, ovine, canine, feline, primate, murine, caprine, or other mammal.

13. The method of claim 10, which is used to produce a cloned cow.

14. A method for producing an avian from ES cells comprising:
(i) isolating ES cells from an avian having desired characteristics;
(ii) expanding said ES cells in culture and optionally cryopreserving said expanded ES cells;
(iii) obtaining eggs that are unable to develop into an embryo;
(iv) injecting said eggs with said ES cells; and
(v) incubating said eggs to produce avian offspring having the genotype of ES cells.

15. The method of claim 13, wherein the avian is a chicken, turkey, guinea hen, ostrich, eagle, osprey, condor, bird of prey, or avian near extinction.

16. The method of any one of claims 7 through 14, wherein the ES cell is genetically modified to express a desired gene(s).


SEQUENCE LISTING (2 sequences)

SEQ ID NO: 1 - DNA, 21 bp, Artificial Sequence
  Description of Artificial Sequence Primer
cgctgtggtacacgctgtgcg

SEQ ID NO: 2 - DNA, 22 bp, Artificial Sequence
  Description of Artificial Sequence Primer
caccatccagtgcaggagctcg